(19) United States
(12) Patent Application Publication
(10) Pub. No.: US 2010/0222308 A1
(43) Pub. Date: Sep. 2, 2010

(54) OPHTHALMIC, OTIC OR NASAL PHARMACEUTICAL COMPOSITION AND THE USE THEREOF

(75) Inventor: Jing Zhang, Shenzhen (CN)

Correspondence Address: STEPTOE & JOHNSON LLP, 2121 AVENUE OF THE STARS, SUITE 2800, LOS ANGELES, CA, 90067, US

(73) Assignee: SHENZHEN REGOO LABORATORIES CO., LTD., Shenzhen, PR (CN)

(21) Appl. No.: 12/663,264

(22) PCT Filed: May 8, 2008
(86) PCT No.: PCT/CN08/00911

§ 371 Date: Dec. 4, 2009

(30) Foreign Application Priority Data
Jun. 6, 2007 (CN) 200710100237.7

(51) Int. Cl. A61K 31/56; A61P 27/02; A61P 31/04; A61P 29/00

(52) U.S. Cl. 514/171

(57) ABSTRACT

The invention provides an ophthalmic, otic or nasal pharmaceutical composition, comprising levofloxacin or the pharmaceutical acceptable salts thereof and loteprednol etabonate, wherein the weight ratio of loteprednol etabonate to levofloxacin is 1:0.2-5. The use of ophthalmic, otic or nasal pharmaceutical composition of the invention in preparation of the medication for treatment of conjunctivitis, keratitis, blepharitis, dacrycystitis, hordeolum, corneal ulcer and ocular infection accompanied with ophthalmitis and even inflammation of the surrounding tissues, to prevent increase of bacterial infection risks and the tissue inflammation of the infected area after the ophthalmic surgeries or ocular injuries, to treat or alleviate the bacterial infection in combination with the tissue inflammation of the infected area, or to treat tympanitis, otitis externa and infective rhinitis.

### FIELD OF THE INVENTION

The invention relates to an ophthalmic, otic or nasal pharmaceutical composition. More particularly, it relates to a pharmaceutical composition comprising levofloxacin or the pharmaceutical acceptable salts thereof and loteprednol etabonate. The invention also relates to the preparation and use of such a composition.

### BACKGROUND OF THE INVENTION

Ocular infection is frequently accompanied with ophthalmitis and even inflammation of the surrounding tissues. Ophthalmic surgeries or ocular injuries often raise up the risk of bacterial infection and cause tissue inflammation of the infected area. Therefore, there needs a compound preparation that is formulated in a single dosage form by one or more antibiotics with an anti-infectious utility and one or more steroids or non-steroids anti-inflammatory agents with an anti-inflammatory function.

Loteprednol etabonate tobramycin eye drops Zylet are US marketed medicine, whose active ingredients are 5 mg loteprednol etabonate (0.5%) and 3 mg tobramycin (0.3%).

Since quinolones have a broader antibiogram than tobramycin, the combination of quinolone drugs and anti-inflammatory agents is more preferable.

Chinese patent application (Publication NO. CN1320035) disclosed that a pharmaceutical composition formulated with quinolone antibiotics i.e. moxifloxacin and steroid anti-inflammatory agents was used to treat ophthalmic diseases. In the application, it was mentioned that the steroid anti-inflammatory agents included loteprednol, however the detailed procedure was not revealed.

Levofloxacin, whose chemical name is (−)-(s)-9-fluoro-2,3-dihydro-3-methyl-10-(4-methyl-1-piperazinyl)-7-oxo-7H-pyrido[1,2,3-de]-benzoxazine-6-carboxylic acid hemihydrate, is a quinolone-type antibacterial, is a levoisomer of ofloxacin, and possesses a broad spectrum Gram-positive bacterium and Gram-negative bacterium resistant activity. There already exists some levofloxacin-containing products on the market, including levofloxacin-containing eye drops. For example, the 1.5% levofloxacin-containing eye drops (product name: Iquix) from Santen co., Japan are on US market, which are used to treat bacterial corneal ulcer; and the eye drops containing 0.5% and 0.3% levofloxacin are on Chinese market, which are used to treat external ocular infection such as bacterial conjunctivitis, keratitis, corneal ulcer, dacrycystitis, and post-surgical infection.

Loteprednol etabonate with the following structure,

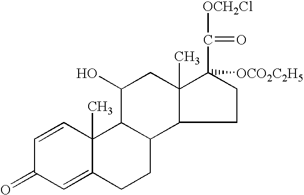

whose chemical name is chloromethyl 17α-[(ethoxycarbonyl)oxy]-11β-hydroxy-3-oxoandrosta-1,4-diene-17β-carboxylate, is steroid-type glucocorticoid with a good anti-inflammatory function. There also has some loteprednol-containing products on the market, including loteprednol-containing ophthalmic suspension, e.g. Alrex and Lotemax approved by FDA of the United States in May, 1998. Loteprednol etabonate, an soft drug which is metabolized into an inactive product rapidly after functioning in vivo, has more safety than general corticosteroid.

Levofloxacin, a quinolone-type broad-spectrum antibiotic with identified pharmaceutical functions, does not significantly irritate eyes during clinic uses. And loteprednol etabonate is suitable to treat steroid response inflammation. So the associated utilization of both is extremely needed in clinic.

Chinese patent application (Publication NO. CN1320035) disclosed a pharmaceutical composition formulated with a quinolone-type antibiotic i.e. moxifloxacin and a steroid anti-inflammatory agent was used to treat ophthalmic diseases, wherein said steroid anti-inflammatory agent includes loteprednol. Nevertheless, a particular composition containing loteprednol etabonate has not been disclosed yet, and the formulation ratio between moxifloxacin and loteprednol etabonate has not been indicated either.

Liping Zhang et al. disclosed a composition comprising ofloxacin and dexamethasone in The research and preparation of compound ofloxacin eye drops and the observation for their curative effects, Jiangsu Pharmacology and Clinic Research, 1999, Vol. 7, 3^{rd} Issue, p 35-36. However, the chemical and physical properties of dexamethasone and loteprednol etabonate are not comparable. So the performance of combining levofloxacin and loteprednol etabonate can not be presumed accordingly.

The inventor surprisedly found that levofloxacin and loteprednol etabonate combined in an ophthalmic, otic or nasal pharmaceutical composition not only provided a synergetic action to effectively treat various inflammations, but alsoslightly lowered ocular pressure, thereby eliminating the side effect of increasing ocular pressure caused by steroid-type anti -inflammatory agents.

### DISCLOSURE OF THE INVENTION

One objective of the invention is to provide an ophthalmic, otic or nasal pharmaceutical composition comprising levofloxacin and loteprednol etabonate, which is used for effectively treating ocular infection accompanied with ophthalmitis and even inflammation of the surrounding tissues, preventing increase of bacterial infection risks and the tissue inflammation of the infected area after ophthalmic surgeries or ocular injuries, and further treating or alleviating the bacterial infection in combination with the tissue inflammation of the infected area, or treating tympanitis, otitis externa and infective rhinitis.

The invention provides an ophthalmic, otic or nasal pharmaceutical composition, comprising levofloxacin or the pharmaceutical acceptable salts thereof and loteprednol etabonate, wherein the weight ratio of loteprednol etabonate to levofloxacin is 1:0.2-5.

According to the invention, the weight ratio between loteprednol etabonate and levofloxacin or the pharmaceutical acceptable salts thereof is preferably 1:0.5-1.75, and more preferably 1:1.

The ophthalmic, otic or nasal pharmaceutical composition of the invention can be in a form of medication selected from a group consisting of eye drops, suspension-type eye drops, eye gel, eye cream, ear drops, and nose drops, whernin suspension-type eye drops are preferable. The ophthalmic, otic or nasal pharmaceutical composition of the invention further comprises one or more pharmaceutical auxiliaries selected from a group consisting of an isotonic regulator, a preservative, a pH regulator, a suspending agent, and a moistening agent.

In a preferable embodiment of the invention, the formulation of suspension-type eye drops, eye gel, eye cream, ear drops, or nose drops is used, wherein there contains 0.1-1.0 g loteprednol etabonate and 0.1-1.0 g levofloxacin or the pharmaceutical acceptable salts thereof per 100 ml preparation, and preferably 0.5 g loteprednol etabonate and 0.5 g levofloxacin or the pharmaceutical acceptable salts thereof per 100 ml preparation.

In a preferable embodiment of the invention, the formulation of suspension-type eye drops is used, wherein there contains 0.5 g loteprednol etabonate, 0.5 g levofloxacin, 0.1-1.5 g boric acid, 0.05-1.7 g borax, 0.01-0.9 g sodium chloride, 0.01-0.5 g poloxamer, 0.01-0.6 g trichloro-tertiary butyl alcohol, 0.01-1 g sodium hyaluronate in each 100 ml eye drops; and the balance is water for injection.

In a more preferable embodiment of the invention, the formulation of suspension-type eye drops is used, wherein there contains 0.5 g loteprednol etabonate, 0.5 g levofloxacin, 1.2 g boric acid, 0.06 g borax, 0.22 g sodium chloride, 0.05 g poloxamer, 0.3 g trichloro-tertiary butyl alcohol, 0.035 g sodium hyaluronate in each 100 ml eye drops, and the balance is water for injection.

The invention also relates to the use of the ophthalmic, otic or nasal pharmaceutical composition described above in preparation of medicines for treatment of conjunctivitis, keratitis, blepharitis, dacrycystitis, hordeolum, corneal ulcer and ocular infection accompanied with ophthalmitis and even inflammation of the surrounding tissues, to prevent increase of bacterial infection risks and the tissue inflammation of the infected area after ophthalmic surgeries or ocular injuries, to treat or alleviate the bacteria infection in combination with the tissue inflammation of the infected area, or to treat tympanitis, otitis externa and infective rhinitis.

In the present invention, the pharmaceutical acceptable salts of levofloxacin preferably is hydrochlorate, lactate, or methane-sulfonate of levofloxacin.

The examples of ophthalmic, otic or nasal conditions which can be treated or prevented by the invention include conjunctivitis, keratitis, blepharitis, dacrycystitis, hordeolum and corneal ulcer. The pharmaceutical composition of the invention may also be prophylactically applied in the cases of the ophthalmic surgeries or ocular injuries possibly causing the above ophthalmic conditions, or tympanitis, otitis externa and infective rhinitis.

For ocular administration, the pharmaceutical composition of the invention is specifically formulated, preferably is sterile, and possesses physical properties, such as isotonic concentration and pH value suitable for ocular administration.

The mentioned levofloxacin was purchased, while loteprednol etabonate was synthesized by Shenzhen Regoo Laboratories Co., Ltd independently.

In the present invention, the eye drops containing levofloxacin and loteprednol etabonate combined in 9 different concentration ratios were tested for their functions of anti-infection and anti-inflammation in a pharmacodynamical prescription screening. The experiment was performed with trabeculotomy, extracapsular extraction and artificial lens implantation simulating clinic treatment for glaucoma, cataract; and incisions simulating clinic surgeries at conjunctiva and sclera, into which Bacillus pyocyaneus was injected to creat a post-surgical infection model. Pharmaceutical solutions with different concentration were dropped into eyelids of model rabbits according to a clinic dosage for continuously 7 days, 4 times/day, 2 drops each time. The results indicates that when the weight ratio between loteprednol etabonate and levofloxacin is 1:0.2-5, preferably 1:0.5-1.75, the composition comprising levofloxacin and loteprednol etabonate has anti-infectious and anti-inflammatory functions. Most preferably, the composition with a weight ratio of 1:1, containing 0.5 g loteprednol etabonate and 0.5 g levofloxacin per 100 ml eye drops, has the most significant pharmaceutical effects in a comprehensive evaluation about 4 indexes of decreasing redness, edema, canthal secretion and lens opacities classification, and results in the least bacterial number in conjunctival bacterial culture.

In particular, the pharmaceutical composition of the invention can be formulated into suspension-type eye drops, or other preparations suitable for ocular administration (such as eye gel, eye cream, etc.).

During the preparation of suspension-type eye drops, it needs to considerate the pH value, osmotic pressure and properties etc. necessary for general eye drops. And the re-dispersivity and sedimentation volume ratio are also required to be taken into account.

The supplements commonly used in suspension-type eye drops mainly include an isotonic regulator, a preservative, a pH regulator. A suspending agent and a moistening agent are also needed to be added into suspension-type eye drops.

An isotonic regulator may be glucose (10-100 g/1000 ml) or sodium chloride (0.1-9 g/1000 ml); a pH regulator may be sodium biphosphate (0.4-10 g/1000 ml) and disodium hydrogen phosphate (0.9-10 g/1000 ml), also may be boric acid (1-15 g/1000 ml) and borax (0.5-17 g/1000 ml); a moistening agent may be Tween-80 (0.1-20 g/1000 ml) or poloxamer (0.1-5 g/1000 ml); a preservative may be ethyl p-hydroxybenzoate (0.1-3.0 g/1000 ml), trichloro-tertiary butyl alcohol (0.1-6 g/1000 ml), benzalkonium bromide (0.1-1 g/1000 ml), benzalkonium chloride (0.1-1 g/1000 ml) or disodium ethylene diamine tetraacetate (0.05-0.2 g/1000 ml); a thickening agent may be glycerol (2-200 g/1000 ml), carbomer (0.1-20 g/1000 ml), methylcellulose (1-30 g/1000 ml), povidone (1-50 g/1000 ml), hydroxy propyl methylcellulose (1-50 g/1000 ml), sodium carboxymethylcellulose (1-50 g/1000 ml) or sodium hyaluronate (0.1-10 g/1000 ml).

In one example of the invention, the pharmaceutical composition was examined regarding to 4 indexes: property, pH value, sedimentation volume ratio and re-dispersivity. The optimal weight ratio of such a pharmaceutical composition is identified, i.e. 0.5% loteprednol, 0.5% levofloxacin, 1.2% boric acid, 0.06% borax, 0.22% sodium chloride, 0.05% poloxamer, 0.3% trichloro-tertiary butyl alcohol, 0.035% sodium hyaluronate, and the balance is water for injection.

Such a prescription is a light yellow-green suspension, with a pH value of 6.1, a sedimentation volume ratio of 0.95, and relatively good optical re-dispersivity.

Preferably, the pharmaceutical composition of the invention is manufactured into suspension-type eye drops comprising levofloxacin (0.5%) and loteprednol (0.5%) (weight/volume (g/ml)). A variety of sizes can be made as needed, for instance, 2.5 ml eye drops in a 5 ml container, 5 ml eye drops in a 8 ml or 10 ml container, and 10 ml eye drops in a 10 ml container, etc.

The process for preparing the pharmaceutical composition described in the invention into suspension-type eye drops is shown as follows:

(1) sterilizing and preparing vials;

(2) crushed and filtering crystallization loteprednol etabonate powder through a 200 mesh and sterilizing by radiation;

(3) dissolving each excipient except loteprednol etabonate and levofloxacin into an 80% amount of water for injection, and after the resultant solution becoming clear, dissolving levofloxacin into the solution to form a yellow-green solution;

(4) filtering the resultant solution through a micropore filter membrane, and preparing to use after sterilization;

(5) dispersing the sterilized loteprednol etabonate in the ready-to-use solution in a hundred grades purification area, and dispersing the resultant sample uniformly by ultrasonic or high-speed shearing;

(7) mixing and packing the eligible solution in a sterile condition, and obtaining the finished products;

(8) testing the finished products.

### DETAILED EMBODIMENTS

The present invention is further explained and illustrated by the following examples which are not to be construed to limit the scope of the invention.

### Example 1

### The Pharmacodynamical Prescription Screening Experiment of Levofloxacin Loteprednol Etabonate Eye Drops with a Series of Concentrations in Rabbits

1. Purpose of the experiment to observe the function of pharmaceutical solutions with different concentration ratios of levofloxacin: loteprednol etabonate eye drops in anti-infection, antibacterial and anti-inflammation following surgeries, and to compare the pharmacodynamical difference between the designed dosages and other dosages, by dropping the pharmaceutical solutions with different ratios into rabbits' eyelids following incision surgery simulation.

2. Experimental Materials:

1) Experimental Samples

Levofloxacin loteprednol etabonate eye drops (LZ01-09), Batch No: 060701.
LZ01: Size: 5 ml: 20 mg loteprednol etabonate+15 mg levofloxacin;
LZ02: Size: 5 ml: 20 mg loteprednol etabonate+25 mg levofloxacin;
LZ03: Size: 5 ml: 20 mg loteprednol etabonate+35 mg levofloxacin;
LZ04: Size: 5 ml: 25 mg loteprednol etabonate+15 mg levofloxacin;
LZ05: Size: 5 ml: 25 mg loteprednol etabonate+25 mg levofloxacin;
LZ06: Size: 5 ml: 25 mg loteprednol etabonate+35 mg levofloxacin;
LZ07: Size: 5 ml: 30 mg loteprednol etabonate+15 mg levofloxacin;
LZ08: Size: 5 ml: 30 mg loteprednol etabonate+25 mg levofloxacin;
LZ09: Size: 5 ml: 30 mg loteprednol etabonate+35 mg levofloxacin.

Levofloxacin loteprednol etabonate eye drops were formulated by the preparation process described above, wherein in the light of the experience in China Pharmacopoeia, the amount of levofloxacin salts was calculated according to the amount of levofloxacin contained therein.

Loteprednol etabonate eye drops, Size: 5 ml: 25 mg loteprednol etabonate, was shorted as Single Prescription L.

Levofloxacin eye drops, Size: 5 ml: 25 mg levofloxacin, was shorted as Single Prescription Z.

2) Experimental Animals

Rabbits: Japanese Big White Ear, 2.0-3.0 kg, Shenyang Shuangyi Laboratory Animal Research Institute, Laboratory Animal Producing Licence No.: 8CXK (Liao)2003-0012, Laboratory Animal utilizing Licence No.: SYXK (Liao)2003-0024.

3) Reagents

Procaine hydrochloride injection, Size: 2 ml: 40 mg, Batch No.: 95-1-3, Jinzhuo 2^{nd} Pharmaceutical factory.

Lidocaine hydrochloride injection, Size: 20 ml: 400 mg, Batch No.: 011201, Shanghai Fuxing Zhaohui Medicine Co., LTD.

Sodium chloride injection, Size: 250 ml: 2.25 g, Batch No.: 200608211, Liaoning Haishen Liansheng Pharmaceutical Co., LTD.

75% alcohol, prepared by diluting 95% (water-free) ethanol.

4) Bacterial Species and Culture Media

Bacillus pyocyaneus (Pseudomonas aeruginosa) was provided by the clinical laboratory in The Second Hospital of China Medical University, with a serial number SJJY-05122210. The bacterial solution was prepared as follows: the preserved Bacillus pyocyaneus was inoculated into a M-H broth media, cultured for 16-18 h at 37° C.; the culture was diluted into a desired concentration with 5% gastric mucosin according to a 10-time series dilution method. (The concentration of Bacillus pyocyaneus is 2.5×10⁴ bacteria/ml).

Culture media: M-H agar media, Batch No. 001116; M-H broth media: Batch No. 000515, provided by National Institute for The control of Pharmaceutical and Biological Products.

4) Equipments and Experimental Appliances

A hand-hold slit lamp: made in Germany

An eye expander, an ophthalmic scalpel, and ophthalmic forceps.

5) Experimental condition: normal and animal rooms, a 12 h day-night switch interval, temperature of 18-24° C., relative moisture of 40-70%.

3. Experimental Methods

Both eyes of each of the above healthy rabbits were examined within 24 h before the experiment. After the examination, 65 rabbits without irritated ocular symptom, corneal defection and conjunctival damage were selected and divided into 13 groups (LZ01-09 dosage group, L5 dosage group, Z5 dosage group, model control group, negative control group, respectively), 5 rabbits/group. Following the fixation of the rabbits, 5 drops of 1% procaine hydrochloride injection solution were dropped into each eye, and 0.1 ml 1% lidocaine hydrochloride injection solution were injected into ciliary muscles. Eyeballs were pressed gently so as to intenerate the eyeball tissues following sufficient anaesthetization. Eyelids were expanded with a sterilized eye expander, and a “1” shape incision with a length of 3-5 mm was cut at the base of corneal edge, so as to show a small amount of aqueous fluid. The same procedure was performed to the other eye. After the surgery, 0.05 ml bacterial solution was injected into the conjunctival layer at the side of the surgical incision by a 0.25 ml syringe with a 4# needle, so that each eye of the rabbit was inoculated with 5×10⁴ Bacillus pyocyaneus/ml. Twenty-four hours after the surgery, medication of each group was dropped into eyes, and the dosage was 2 drops/time, 4 times/day (approximately at 8, 11, 14, 17 o'clock), for continuously 7 days; while excipient with the same volume was given to the negative control. The surgical parts and conjunctiva were observed with a hand-hold slit lamp before the first administration each day. The observation indexes included: whether the surgical parts showed redness; whether cornea and conjunctiva was edematous; whether there was secretion at canthi; whether the lens was turbid, and the detailed scoring standard was shown as the following table. The animals were sacrificed by euthanasia 8 days after administration, and ocular conjunctiva was extracted. The conjunctival slice at the surgical part was drilled out by a ring driller with a diameter of 1 cm, cut, and placed into a tube containing 10 ml sodium chloride injection solution. One milliliter of each sample was added into two regular agar media respectively and cultured in a 35° C. incubator for 48 h. The number of colonies was counted, and a t-test was carried out with the results.

Attached Table: Ocular symptoms

| | | Score |
|---|---|---|
| A. | redness (surgery parts and palpebral conjunctiva, | |
| | bulbal conjunctiva parts) | |
| | normal blood vessels, normal color | 0 |
| | congested and redish blood vessels, red and slightly | 1 |
| | evaginated wound | |
| | congested and redish blood vessels, undistinguishable | 2 |
| | blood vessels, evaginated and protuberant wound | |
| | fuchsia diffuse congest, severely evaginated and | 3 |
| | protuberant wound | |
| B. | edema | |
| | no edema | 0 |
| | slight edema (including nictitating membrane) | 1 |
| | apparent edema, accompanied with partially | 2 |
| | evaginated eyelids | |
| | edema to almost half-way closure of eyelid | 3 |
| C. | secretion | |
| | no secretion | 0 |
| | a small amount of secretion | 1 |
| | moist or sticky eyelid and eyelash caused by secretion | 2 |
| | moist or sticky eye area caused by secretion | 3 |
| D. | turbid cornea (based on the most condensed part): | |
| | no turbidity | 0 |
| | dispersed or diffuse turbidity, clearly observable iris | 1 |
| | distinguishable semitransparent area, blur iris | 2 |
| | showing canous transparent area, blur iris, barely seeing | 3 |
| | size of pupil | |

| | |
|---|---|
| Total score: | 12 |

4. Results and Conclusion

4.1 Results of the Daily Observation and Scoring

Observation by a hand-hold slit lamp and scoring in accordance with the attached table were carried out before daily administration since 24 h after the surgery. whether the surgical parts showed redness; whether cornea and conjunctiva was edematous; whether there was secretion at canthi; and whether the lens was turbid were scored, and scoring results of those 4 indexes and in total were shown in the following table. The scoring results were based on statistical scoring variation rate (W %), which was calculated by the following formula:

W%=(Wₙ−W₁)/W₁×100%
W₁: the score at Day 1: Wₙ: the score at Day n.
   (1) Redness symptom at the surgical part: Surgeries of glaucoma and cataract usually employ trabeculotomy, extracapsular extraction and artificial lens implantation. A longitudinal and deep incision was made at conjunctiva and sclera of eyes, and the redness of the surgical incision and congestive symptom in eyes represented the post-surgical infection degree. That index was used to score congestion and redness of ocular surgical incision and eyelids. The healing degree of the incision was observed.

TABLE 1
The redness symptom score during administration of LZ eye drops with different
concentration and single prescription for 7 days at the surgical part (n = 10)

| | Ocular redness score of | | | | | | |
|---|---|---|---|---|---|---|---|
| | experimental rabbits ( X̅ ± SD) | | | | | | |
| Group | 1^{st} day | 2^{nd} day | 3^{rd} day | 4^{th} day | 5^{th} day | 6^{th} day | 7^{th} day |
| LZ01 | 2.6 ± 0.52 | 2.7 ± 0.48 | 2.0 ± 0.67** | 1.9 ± 0.88** | 2.0 ± 0.67 | 2.0 ± 1.05 | 1.9 ± 1.10 |
| LZ02 | 2.6 ± 0.52 | 2.8 ± 0.42 | 2.5 ± 0.53 | 2.4 ± 0.70 | 2.5 ± 0.97 | 1.7 ± 1.06 | 1.6 ± 1.07** |
| LZ03 | 3.0 ± 0.00 | 2.5 ± 0.53 | 2.3 ± 0.48 | 2.1 ± 1.10 | 2.2 ± 1.03 | 1.5 ± 0.85** | 1.3 ± 0.95** |
| LZ04 | 2.9 ± 0.32 | 2.8 ± 0.42 | 2.5 ± 0.71 | 2.1 ± 0.88 | 2.0 ± 1.25 | 1.8 ± 1.03 | 1.4 ± 0.84** |
| LZ05 | 2.8 ± 0.42 | 2.6 ± 0.52 | 2.3 ± 0.82 | 2.2 ± 0.48 | 1.7 ± 1.25 | 1.7 ± 1.06 | 1.2 ± 0.79** |
| LZ06 | 2.6 ± 0.70 | 2.8 ± 0.42 | 2.4 ± 0.70 | 1.5 ± 1.27 | 1.5 ± 1.27 | 1.7 ± 1.25 | 1.5 ± 1.27 |
| LZ07 | 2.9 ± 0.32 | 2.6 ± 0.52 | 2.3 ± 0.48 | 1.9 ± 0.88 | 1.8 ± 1.23 | 2.0 ± 0.82 | 1.8 ± 0.79** |
| LZ08 | 3.0 ± 0.00 | 2.8 ± 0.42 | 2.6 ± 0.52 | 2.5 ± 0.07 | 2.5 ± 0.97 | 2.4 ± 0.97 | 1.9 ± 0.88 |
| LZ09 | 2.5 ± 0.71 | 2.6 ± 0.52 | 2.4 ± 0.84 | 2.3 ± 1.06 | 2.1 ± 0.99 | 1.9 ± 1.20 | 1.8 ± 1.32 |
| L | 3.0 ± 0.00 | 2.8 ± 0.42 | 2.8 ± 0.63 | 2.6 ± 0.97 | 2.7 ± 0.67 | 2.6 ± 0.70 | 2.6 ± 0.70 |
| Z | 2.8 ± 0.42 | 2.8 ± 0.42 | 2.5 ± 0.71 | 2.4 ± 0.84 | 1.9 ± 1.10 | 2.2 ± 1.14 | 2.0 ± 1.05 |
| Model | 2.6 ± 0.52^{ΔΔ} | 2.8 ± 0.42^{ΔΔ} | 2.8 ± 0.42^{ΔΔ} | 2.9 ± 0.32^{ΔΔ} | 2.9 ± 0.32^{ΔΔ} | 2.8 ± 0.42^{ΔΔ} | 2.8 ± 0.42^{ΔΔ} |
| Control | 0.1 ± 0.32 | 0.0 ± 0.00 | 0.0 ± 0.00 | 0.0 ± 0.00 | 0.0 ± 0.00 | 0.0 ± 0.00 | 0.0 ± 0.00 |

**P < 0.01 vs. the model group;
^{ΔΔ}P < 0.01 vs. the control group

As shown in Table 1, the various LZ prescriptions started to show an apparent pharmacodynamical effect on the redness symptom at the surgical part since Day 3 post-administration. After a recovery stage of Day 4-6, it was observed that at Day 7 post-administration, the surgical parts almost healed up, showing no apparent redness or congestive symptom, and the scores other than those in the model group were significantly different than those in the model group at the same stage. The prescriptions with the highest ocular redness scoring variation rate were LZ05 (57.1%), LZ03 (56.7%) and LZ04 (51.7%), showing clearly reduced or eliminated redness symptom, complete healing or almost reaching a normal level at the wound compared with the model group.

4.2 Edema Symptoms at Cornea and Conjunctiva:

Post-surgical infection would cause an edema symptom at ocular cornea and conjunctival. The observation of edema symptom after administration indicated the degree of ocular inflammatory responses.

TABLE 2
The edema symptom scores during administration of LZ eye drops with different
concentration and single prescription for 7 days at cornea and conjunctival (n = 10)

| | Ocular edema score of | | | | | | |
|---|---|---|---|---|---|---|---|
| | experimental rabbits ( X̅ ± SD) | | | | | | |
| Group | 1^{st} day | 2^{nd} day | 3^{rd} day | 4^{th} day | 5^{th} day | 6^{th} day | 7^{th} day |
| LZ01 | 2.9 ± 0.32 | 2.9 ± 0.32 | 2.3 ± 0.48** | 2.2 ± 0.79 | 2.1 ± 0.74 | 2.1 ± 0.57** | 1.7 ± 0.82** |
| LZ02 | 3.0 ± 0.00 | 2.9 ± 0.32 | 2.4 ± 0.70 | 2.5 ± 0.53 | 2.6 ± 0.97 | 2.0 ± 0.94 | 1.8 ± 1.03 |
| LZ03 | 3.0 ± 0.00 | 2.9 ± 0.32 | 2.9 ± 0.32 | 2.1 ± 1.10 | 2.1 ± 1.10 | 2.0 ± 1.05 | 1.8 ± 1.03 |
| LZ04 | 2.9 ± 0.32 | 3.0 ± 0.00 | 2.2 ± 0.63** | 1.7 ± 0.67** | 2.0 ± 1.25 | 2.1 ± 0.99 | 1.5 ± 0.85** |
| LZ05 | 2.9 ± 0.32 | 2.7 ± 0.48 | 2.5 ± 0.53 | 2.3 ± 0.82 | 1.8 ± 1.23 | 1.6 ± 1.07** | 1.4 ± 0.84** |
| LZ06 | 2.8 ± 0.42 | 2.8 ± 0.42 | 2.4 ± 0.70 | 1.4 ± 1.17** | 1.3 ± 1.34** | 1.6 ± 1.26 | 1.4 ± 1.26** |
| LZ07 | 3.0 ± 0.00 | 3.0 ± 0.00 | 2.2 ± 0.42** | 1.8 ± 0.79** | 1.7 ± 1.25 | 2.0 ± 0.94 | 1.7 ± 0.95** |
| LZ08 | 3.0 ± 0.00 | 3.0 ± 0.00 | 2.7 ± 0.67 | 2.5 ± 0.97 | 2.4 ± 1.07 | 2.4 ± 0.97 | 1.9 ± 0.88 |
| LZ09 | 2.6 ± 0.52 | 2.9 ± 0.32 | 2.3 ± 0.67 | 2.0 ± 1.05 | 2.1 ± 1.10 | 2.0 ± 1.15 | 1.8 ± 1.14 |
| L | 2.9 ± 0.32 | 2.9 ± 0.32 | 2.6 ± 0.70 | 2.6 ± 0.97 | 2.6 ± 0.97 | 2.7 ± 0.48 | 2.6 ± 0.70 |
| Z | 2.6 ± 0.84 | 2.8 ± 0.42 | 2.6 ± 0.52 | 2.4 ± 0.84 | 1.9 ± 1.10 | 2.2 ± 0.92 | 2.2 ± 0.92 |
| Model | 2.7 ± 0.48^{ΔΔ} | 2.9 ± 0.32^{ΔΔ} | 2.9 ± 0.32^{ΔΔ} | 2.8 ± 0.42^{ΔΔ} | 2.8 ± 0.42^{ΔΔ} | 2.8 ± 0.42^{ΔΔ} | 2.8 ± 0.42^{ΔΔ} |
| Control | 0.0 ± 0.00 | 0.0 ± 0.00 | 0.0 ± 0.00 | 0.0 ± 0.00 | 0.1 ± 0.32 | 0.0 ± 0.00 | 0.0 ± 0.00 |

**P < 0.01 vs. the model group;
^{ΔΔ}P < 0.01 vs. the control group

As shown in Table 2, the various LZ prescriptions started to alleviate the edema symptom at cornea and conjunctival since Day 3 post-administration. At Day 7 post-administration, each prescription-administrated group (except for a few animals) showed a clearly alleviated edema symptom, and the probability of intra-ocular pressure increase could be reduced by loteprednol etabonate. Therefore, severity of the edema symptom was relatively low, and the score showed a significant difference from that of the model group (P<0.01). The prescriptions with higher edema scoring variation rate were LZ05 (51.7%), LZ06 (50.0%) and LZ04 (48.3%), which showed significantly alleviated or eliminated corneal and conjunctival edema symptom, while compared with the model group.

### 4.3 Canthal Secretion:

Observation of an inflammatory response after ocular infection, the status of canthal secretion, could be used to assist in determining the degree of ocular inflammatory responses.

TABLE 3
The canthal secretion scores during administration of LZ eye drops with different
concentration and single prescription for 7 days (n = 10)

| | Ocular edema score of experimental rabbits ( X̅ ± SD) | | | | | | |
|---|---|---|---|---|---|---|---|
| Group | 1^{st} day | 2^{nd} day | 3^{rd} day | 4^{th} day | 5^{th} day | 6^{th} day | 7^{th} day |
| LZ01 | 2.0 ± 0.94 | 1.7 ± 0.82** | 1.5 ± 0.71** | 1.2 ± 1.14** | 1.0 ± 0.94** | 0.7 ± 1.06** | 0.5 ± 0.71** |
| LZ02 | 2.5 ± 0.85 | 2.6 ± 0.70 | 2.3 ± 0.67 | 2.2 ± 1.03 | 1.9 ± 0.99 | 1.3 ± 0.67 | 0.8 ± 0.79 |
| LZ03 | 2.6 ± 0.52 | 1.8 ± 0.79 | 2.0 ± 0.47 | 1.4 ± 1.84** | 1.5 ± 0.97 | 1.0 ± 1.15 | 0.8 ± 0.92 |
| LZ04 | 2.2 ± 0.92 | 1.4 ± 0.70** | 1.5 ± 0.71** | 0.8 ± 1.03** | 1.7 ± 1.16 | 0.9 ± 0.88** | 0.4 ± 0.52** |
| LZ05 | 2.2 ± 1.03 | 1.9 ± 0.88 | 1.6 ± 0.52** | 0.9 ± 0.57** | 0.9 ± 0.99** | 0.7 ± 0.67** | 0.3 ± 0.48** |
| LZ06 | 1.8 ± 0.79 | 2.1 ± 0.88 | 1.7 ± 0.95 | 1.2 ± 1.32** | 0.8 ± 0.92** | 0.9 ± 0.74** | 0.4 ± 0.52** |
| LZ07 | 2.2 ± 0.92 | 2.3 ± 0.82 | 1.9 ± 0.74 | 1.5 ± 0.97** | 1.4 ± 1.07 | 1.3 ± 1.16 | 0.8 ± 0.79 |
| LZ08 | 2.4 ± 0.70 | 2.2 ± 0.92 | 2.0 ± 0.94 | 1.5 ± 1.27 | 1.4 ± 0.97** | 1.3 ± 1.16 | 0.8 ± 0.79 |
| LZ09 | 2.0 ± 0.94 | 2.1 ± 0.99 | 2.0 ± 0.94 | 1.7 ± 1.06 | 1.7 ± 1.25 | 1.4 ± 0.97 | 0.7 ± 0.82 |
| L | 2.2 ± 0.79 | 2.7 ± 0.67 | 2.7 ± 0.67 | 2.4 ± 1.07 | 2.1 ± 1.10 | 2.0 ± 0.94 | 1.4 ± 0.97 |
| Z | 2.2 ± 0.92 | 2.3 ± 0.82 | 1.9 ± 0.88 | 1.6 ± 1.17 | 1.2 ± 0.79** | 1.3 ± 1.06 | 0.9 ± 1.10 |
| Model | 2.8 ± 0.42^{ΔΔ} | 2.7 ± 0.67^{ΔΔ} | 2.7 ± 0.67^{ΔΔ} | 2.6 ± 0.52^{ΔΔ} | 2.6 ± 0.84^{ΔΔ} | 2.1 ± 0.88^{ΔΔ} | 1.4 ± 0.52^{ΔΔ} |
| Control | 0.3 ± 0.48 | 0.2 ± 0.42 | 0.2 ± 0.42 | 0.2 ± 0.42 | 0.2 ± 0.42 | 0.0 ± 0.00 | 0.1 ± 0.32 |

**P < 0.01 vs. the model group;
^{ΔΔ}P < 0.01 vs. the control group

As shown in Table 3, the various LZ prescriptions started to reduce canthal secretion since Day 2 post-administration. At Day 7 post-administration, ocular secretion almost disappeared in each prescription group, showing no apparent difference from the negative control group. Secretion was not eliminated in only a few animals. The scores starting from Day 3 showed a significant difference from those of the model group at the same stage (P<0.01). The prescriptions with higher canthal secretion scoring variation rate were LZ05 (86.4%), LZ04 (81.8%) and LZ06 (77.8%), showing clearly reduced or eliminated canthal secretion while compared with the model group.

4.4 Ocular Lens Opacities Classification:

Ocular inflammatory responses would resulted in fullness of inflammatory medium in lenses and opacities and darkness of their surface. In addition, Bacillus pyocyaneus was injected into the surgical incision, so that the condensed inflammatory solution caused lenses to be relatively opacities under a slit lamp. The opacities classification of lenses was used to determine the weakened degree and trend of Bacillus pyocyaneus-caused inflammatory responses.

TABLE 4
The lens opacities classification scores during administration of LZ eye drops with
different concentration and single prescription for 7 days (n = 10)

| | Ocular lens opacities classification score of experimental rabbits ( X̅ ± SD) | | | | | | |
|---|---|---|---|---|---|---|---|
| Group | 1^{st} day | 2^{nd} day | 3^{rd} day | 4^{th} day | 5^{th} day | 6^{th} day | 7^{th} day |
| LZ01 | 2.8 ± 0.42 | 2.7 ± 0.48 | 2.6 ± 0.52 | 2.0 ± 0.82 | 2.6 ± 0.52 | 2.5 ± 0.53 | 2.0 ± 0.94 |
| LZ02 | 3.0 ± 0.00 | 2.9 ± 0.32 | 2.7 ± 0.67 | 2.6 ± 0.70 | 2.2 ± 1.32 | 2.6 ± 0.70 | 2.3 ± 0.95 |
| LZ03 | 2.9 ± 0.32 | 2.8 ± 0.63 | 2.9 ± 0.32 | 2.0 ± 1.25 | 2.1 ± 1.10 | 1.9 ± 1.45 | 1.7 ± 1.34 |
| LZ04 | 2.7 ± 0.67 | 3.0 ± 0.00 | 2.7 ± 0.67 | 2.3 ± 1.06 | 2.0 ± 1.25 | 2.1 ± 1.10 | 1.6 ± 0.84** |
| LZ05 | 2.7 ± 0.67 | 2.8 ± 0.42 | 2.7 ± 0.48 | 2.6 ± 0.53 | 1.9 ± 1.29 | 2.0 ± 1.25 | 1.6 ± 1.07** |
| LZ06 | 2.6 ± 0.84 | 2.7 ± 0.67 | 2.5 ± 0.85 | 1.5 ± 1.43 | 1.6 ± 1.50 | 1.7 ± 1.42 | 1.6 ± 1.35 |
| LZ07 | 2.8 ± 0.42 | 2.9 ± 0.32 | 2.6 ± 0.52 | 2.0 ± 0.94 | 2.0 ± 1.05 | 2.0 ± 1.05 | 1.7 ± 0.95** |
| LZ08 | 2.9 ± 0.32 | 3.0 ± 0.00 | 2.9 ± 0.32 | 2.6 ± 0.97 | 2.5 ± 0.97 | 2.4 ± 1.07 | 2.2 ± 1.03 |
| LZ09 | 2.4 ± 0.97 | 2.8 ± 0.42 | 2.5 ± 0.71 | 2.0 ± 1.15 | 2.1 ± 1.10 | 1.9 ± 1.29 | 1.8 ± 1.23 |
| L | 2.6 ± 0.70 | 2.8 ± 0.63 | 2.8 ± 0.63 | 2.5 ± 1.08 | 2.7 ± 0.95 | 2.7 ± 0.95 | 2.7 ± 0.95 |
| Z | 2.6 ± 0.84 | 2.7 ± 0.67 | 2.4 ± 0.84 | 2.4 ± 0.84 | 2.3 ± 1.06 | 2.3 ± 1.06 | 2.1 ± 1.10 |
| Model | 3.0 ± 0.00^{ΔΔ} | 2.7 ± 0.67^{ΔΔ} | 2.9 ± 0.32^{ΔΔ} | 2.9 ± 0.32^{ΔΔ} | 2.9 ± 0.32^{ΔΔ} | 2.8 ± 0.42^{ΔΔ} | 2.8 ± 0.42^{ΔΔ} |
| Control | 0.1 ± 0.32 | 0.1 ± 0.32 | 0.1 ± 0.32 | 0.0 ± 0.00 | 0.0 ± 0.00 | 0.0 ± 0.00 | 0.0 ± 0.00 |

**P < 0.01 vs. the model group;
^{ΔΔ}P < 0.01 vs. the control group

As shown in Table 4, the various LZ prescriptions started to reduce lens opacities since Day 5-7 post-administration. At Day 7, scores of LZ04, LZ05 and LZ07 showed significant differences from those of the model group (P<0.01). Due to the physiological structure of eyes and lenses, inflammatory medium could not be absorbed and eliminated rapidly. The classification of opacities during a 7-day experimental period would only drop a certain level, but not completely reach the level of negative control animals. The prescriptions with higher distinguishable ocular lens opacities classification scoring variation rate were LZ03 (41.4%), LZ04, LZ05 (40.7%), LZ07 (39.3%) and LZ06 (38.5%), showing a reduced ocular lens opacities, while compared with the model control.

4.5 Integrative Scoring of Ocular Symptoms:

To scoring for whether the surgical ocular parts showed redness; whether cornea and conjunctiva was edematous; whether there was secretion at canthi; whether the ocular lens was opacities, the above 4 scores were summed. It was screened for the better LZ prescriptions by an integrative judgement.

TABLE 5
The ocular symptom total scores during administration of LZ eye drops with
different concentration and single prescription for 7 days (n = 10)

| | Ocular symptom total score of experimental rabbits ( X̅ ± SD) | | | | | | |
|---|---|---|---|---|---|---|---|
| Group | 1^{st} day | 2^{nd} day | 3^{rd} day | 4^{th} day | 5^{th} day | 6^{th} day | 7^{th} day |
| LZ01 | 10.3 ± 1.25 | 10.0 ± 1.49 | 8.4 ± 0.96** | 7.3 ± 3.13** | 7.7 ± 2.26** | 7.3 ± 2.67** | 6.1 ± 3.31** |
| LZ02 | 11.1 ± 1.20 | 11.2 ± 1.55 | 9.9 ± 2.33 | 9.7 ± 2.75 | 9.2 ± 3.49 | 7.6 ± 2.80 | 6.5 ± 3.50 |
| LZ03 | 11.5 ± 0.71 | 10.0 ± 1.82 | 10.1 ± 1.29 | 7.6 ± 4.12 | 7.9 ± 3.93 | 6.4 ± 3.92** | 5.6 ± 3.89** |
| LZ04 | 10.7 ± 1.89 | 10.2 ± 0.92 | 8.9 ± 2.13 | 6.9 ± 3.18** | 7.7 ± 4.83 | 6.9 ± 3.35 | 4.9 ± 2.73** |
| LZ05 | 10.6 ± 1.96 | 10.0 ± 2.00 | 9.1 ± 1.79** | 8.0 ± 1.90** | 6.3 ± 4.37** | 6.0 ± 3.74** | 4.5 ± 2.80** |
| LZ06 | 9.8 ± 2.44 | 10.4 ± 2.17 | 9.0 ± 2.54 | 5.6 ± 4.95** | 5.2 ± 4.85** | 5.9 ± 4.25** | 4.9 ± 4.25** |
| LZ07 | 10.9 ± 1.45 | 10.8 ± 1.48 | 9.0 ± 1.56** | 7.2 ± 3.22** | 6.9 ± 4.33 | 7.3 ± 3.56 | 6.0 ± 3.16** |
| LZ08 | 11.3 ± 0.95 | 11.0 ± 1.15 | 10.2 ± 1.93 | 9.1 ± 3.63 | 8.8 ± 3.71 | 8.5 ± 3.60 | 6.8 ± 3.05 |
| LZ09 | 9.5 ± 2.92 | 10.4 ± 1.78 | 9.2 ± 2.90 | 8.0 ± 4.11 | 8.0 ± 4.14 | 7.2 ± 4.37 | 6.1 ± 4.28 |
| L | 10.7 ± 1.64 | 11.2 ± 1.87 | 10.9 ± 2.51 | 10.1 ± 4.01 | 10.1 ± 3.45 | 10.0 ± 2.79 | 9.3 ± 2.91 |
| Z | 10.2 ± 2.82 | 10.6 ± 2.22 | 9.4 ± 2.55 | 8.8 ± 3.29 | 7.3 ± 3.92 | 8.0 ± 3.68 | 7.2 ± 3.82 |
| Model | 11.1 ± 0.10^{ΔΔ} | 11.1 ± 1.91^{ΔΔ} | 11.3 ± 1.49^{ΔΔ} | 11.2 ± 1.23^{ΔΔ} | 11.2 ± 1.75^{ΔΔ} | 10.5 ± 1.96^{ΔΔ} | 9.8 ± 1.55^{ΔΔ} |
| Control | 0.5 ± 0.53 | 0.3 ± 0.48 | 0.3 ± 0.48 | 0.0 ± 0.00 | 0.3 ± 0.48 | 0.0 ± 0.00 | 0.1 ± 0.32 |

**P < 0.01 vs. the model group;
^{ΔΔ}P < 0.01 vs. the control group

As shown in Table 5, the medication started to affect the total scores of redness, edema, canthal secretion, ocular lens opacities classification evidently since Day 3 post-administration, wherein scores of LZ01, LZ04, LZ05, LZ06, LZ07 showed significant differences from those of the model group (P<0.01). The prescriptions with higher total scoring variation rate were LZ05 (57.5%), LZ04 (54.2%) and LZ03 (51.3%), showing decreased total scores and clearly pharmacodynamical actions.

4.6 The Results of Conjunctival Colonies in the Rabbit Surgical Infection Model

The animals were sacrificed by euthanasia 8 days following administration, and ocular conjunctiva was extracted. The conjunctival slice at the surgical part was drilled out by a ring driller with a diameter of 1 cm, cut, and placed into a tube containing 10 ml sodium chloride injection solution. One milliliter of each sample was added into two regular agar media respectively and cultured in a 35° C. incubator for 48 h. The number of colonies was counted, and the results were shown in Table 6.

TABLE 6
The conjunctival bacterial culture results during administration
of LZ eye drops with different concentration and single
prescription for 7 days (X ± SD)

| Group | Dosage(d/day) | No | No of Bacteria (×10/position) |
|---|---|---|---|
| LZ01 | 8 | 10 | 30.6 ± 17.0** |
| LZ02 | 8 | 10 | 12.0 ± 9.8** |
| LZ03 | 8 | 10 | 25.8 ± 15.7** |
| LZ04 | 8 | 10 | 7.1 ± 5.0** |
| LZ05 | 8 | 10 | 6.3 ± 5.1** |
| LZ06 | 8 | 10 | 11.8 ± 9.3** |
| LZ07 | 8 | 10 | 5.2 ± 3.6** |
| LZ08 | 8 | 10 | 7.6 ± 7.7** |
| LZ09 | 8 | 10 | 19.5 ± 14.5** |
| L | 8 | 10 | 37.3 ± 25.3** |
| Z | 8 | 10 | 10.7 ± 9.4** |
| Model | 8 | 10 | 109.7 ± 76.6^{ΔΔ} |
| Control | 8 | 10 | 0.1 ± 0.4 |

**P < 0.01 vs. the model group;
^{ΔΔ}P < 0.01 vs. the control group

Summary: Though culturing of ocular conjunctival bacteria, it is observed that the prescription medication of LZ series remarkably inhibited Bacillus pyocyaneus-caused surgical infectious inflammatory responses, wherein in 4 prescriptions, i.e. LZ04 (7.1×10 bacterial/lens), LZ05 (6.3×10 bacterial/lens), LZ07 (5.2×10 bacterial/lens) and LZ08 (7.6×10 bacterial/lens), bacterial numbers were less than 10×10 bacteria/lens, which was significantly lower than those of other medication groups and the model group.

5. Conclusion:

In this experiment, therapeutical surgeries of glaucoma and cataract were simulated in rabbit eye models, and 0.05 ml of 2.5×10⁴ Bacillus pyocyaneus/ml was injected into the surgical wound. Each LZ eye drop prescription was administrated continuously for 7 days, 4 times/day, 2 drops/time. All LZ eye drop prescription reduced infection degree, alleviated inflammatory response symptoms, and facilitated healing of the wound at a certain degree. The integrative observation of the 4 items under a slit lamp and the experimental results of conjunctival bacterium culturing showed that the prescription with a more apparent pharmaceutical function was LZ05 (Size 5 ml: 25 mg loteprednol etabonate+25 mg levofloxacin), wherein 1:1 of loteprednol etabonate: levofloxacin were suspended. Such design not only took treatment of loteprednol etabonate steroid reactive inflammation into account, but also constituted a compound preparation together with the broad-spectrum antibacterial levofloxacin, thereby being suitable for anti-infection and anti-inflammatory response after surgeries. In the experimental rabbits, the inflammatory symptom started to be alleviated since Day 3 post-administration and most of the symptoms were essentially eliminated at Day 7. The period of treatment in clinic is 14 days, which has better curative functions and more significant effects on stabilizing the prevention of infection, performing anti-infection and antibacterial actions, and facilitating the healing of the wound.

### Example 2

### Preparation

TABLE 7
Prescriptions (Unit: g/200 ml)

| Raw material and | Prescriptions | | | | | | | | |
|---|---|---|---|---|---|---|---|---|---|
| excipient | I | II | III | IV | V | VI | VII | VIII | single |
| loteprednol etabonate | 1 | 1 | 1 | 1 | 1 | 1 | 1 | 1 | 1 |
| levofloxacin | 1 | 1 | 1 | 1 | 1 | 1 | 1 | 1 | — |
| boric acid | 3.56 | 3.56 | — | — | 2.4 | 2.4 | 2.4 | 2.4 | — |
| borax | — | — | — | — | 0.12 | 0.12 | 0.12 | 0.12 | — |
| Tween-80 | 0.15 | 0.15 | — | — | — | — | — | — | — |
| hydroxy propyl | 1 | 0.6 | — | — | — | — | — | — | — |
| methylcellulose | | | | | | | | | |
| sodium chloride | — | — | 1.8 | 1.8 | 0.44 | 0.44 | 0.44 | 0.44 | — |
| poloxamer | — | — | 0.06 | 0.06 | 0.06 | 0.06 | 0.06 | 0.06 | — |
| benzalkonium | 0.02 | 0.02 | 0.02 | — | — | — | — | — | — |
| bromide | | | | | | | | | |
| trichloro-tertiary | — | — | — | 0.6 | 0.6 | 0.6 | 0.6 | 0.6 | — |
| butyl alcohol | | | | | | | | | |
| ethyl | — | — | — | — | — | — | — | — | — |
| Ethylparaben | | | | | | | | | |
| sodium | — | — | 0.3 | 0.3 | 0.3 | 0.24 | 0.1 | 0.07 | — |
| hyaluronate | | | | | | | | | |
| Povidone K₃₀ | — | — | — | — | — | — | — | — | 1.2 |
| EDTA•2Na | — | — | — | — | — | — | — | — | 0.02 |
| glycerol | — | — | — | — | — | — | — | — | 4.8 |
| benzalkonium | — | — | — | — | — | — | — | — | 0.02 |
| chloride | | | | | | | | | |
| aminocaproic acid | — | — | — | — | — | — | — | — | 0.2 |

Feeding was performed according to the ratio described in above prescriptions.

2. The process for preparing the pharmaceutical composition described in the invention into suspension-type eye drops is shown below:

(1) sterilizing and preparing vials;

(2) crushed and filtering crystallization loteprednol etabonate powder through a 200 mesh sieve and sterilizing by radiation;

(3) dissolving each excipient except loteprednol etabonate and levofloxacin into an 80% amount of water for injection, and after the resultant solution becoming clear, dissolving levofloxacin into the solution to form a yellow-green solution;

(4) filtering the resultant solution through a micropore filter membrane, and preparing to use after sterilization for 30 min with 100° C. flowing steam;

(5) dispersing the sterilized loteprednol etabonate in the resultant solution in a hundred grades purification area, and dispersing the resultant sample uniformly by ultrasonic or high-speed shearing;

(6) examining the intermediate;

(8) mixing and packaging the eligible solution under a sterile condition, thus obtaining the finished products.

3. Results of screening:

It was shown in Table 8.

TABLE 8
Results of screening the prescriptions

| | index | | | |
|---|---|---|---|---|
| | | | sedimentation | |
| prescription | properties | pH | volume ratio | re-dispersivity |
| Prescription | light yellow-green | 5.6 | 0.98 | worse |
| I | suspension | | | |
| Prescription | light yellow-green | 5.6 | 0.96 | worse |
| II | suspension | | | |
| | sodium hyaluronate | — | — | — |
| | and benzalkonium | | | |
| Prescription | bromide form | | | |
| III | floccule-like | | | |
| | precipitate | | | |
| Prescription | light yellow-green | 4.4 | 0.99 | better |
| IV | suspension, | | | |
| | camphor-like odor | | | |
| Prescription | light yellow-green | 6.1 | 0.98 | better |
| V | suspension | | | |
| Prescription | light yellow-green | 6.0 | 0.96 | better |
| VI | suspension | | | |
| Prescription | light yellow-green | 5.9 | 0.95 | better |
| VII | suspension | | | |
| Prescription | light yellow-green | 6.1 | 0.95 | better |
| VIII | suspension | | | |
| Single | white suspension | 5.4 | 0.95 | better |
| Prescription | | | | |

Normal levofloxacin is light yellow-green color.

Among others, sedimentation volume ratio was detected in accordance with Part II in China Pharmacopoeia. Re-dispersivity was evaluated by the following method: the longer the period required to re-form a suspension by being shaken on a shaker after sedimentation, the worse re-dispersivity is.

### Example 3

### The Effect of Levofloxacin Loteprednol Etabonate Compound (Shown as LZ) on Intra-Ocular Pressure of Rabbits

Twenty healthy rabbits were divided into 4 groups randomly (LZ-H: high dosage group, LZ-M: medium dosage group, LZ-L: low dosage group, negative control group, respectively), 5 animals per group. After the eye drops with a LZ05 mixing ratio described in Example 1 were dropped into both eyes, LZ-H, LZ-M, and LZ-L was administrated as 4, 2, and 1 drops, and the 2 drops of menstruum was dropped in the negative control group. The intra-ocular pressure values were measured by a YZ7A intra-ocular manometer at 0 min, 30 min, 60 min, 120 min, 180 min, 240 min, 300 min and 360 min of administration while anaesthetization with dicaine. The above method was found in this reference: The effect of D-timolol on intra-ocular pressure and β-interruption and the dynamic variation of pharmaceutical concentration in ocular humor aquesus, Journal of ocular pharmacology, 1989, 5:271-279. The result of data was shown in Table 9.

TABLE 9
The effect of LZ eye drops on intra-ocular pressure in rabbits (X ± SD)

| | Intra-ocular pressure (Kpa) | | | | | | | |
|---|---|---|---|---|---|---|---|---|
| Group | 0 min | 30 min | 60 min | 120 min | 180 min | 240 min | 300 min | 360 min |
| LZ-H | 2.78 ± 0.5 | 2.69 ± 0.5 | 2.68 ± 0.5 | 2.51 ± 0.3 | 2.44 ± 0.2* | 2.46 ± 0.2* | 2.50 ± 0.2* | 2.46 ± 0.2** |
| LZ-M | 2.74 ± 0.4 | 2.75 ± 0.5 | 2.77 ± 0.4 | 2.64 ± 0.4 | 2.55 ± 0.3 | 2.55 ± 0.3 | 2.53 ± 0.3* | 2.48 ± 0.2* |
| LZ-L | 2.78 ± 0.4 | 2.73 ± 0.3 | 2.75 ± 0.3 | 2.71 ± 0.3 | 2.78 ± 0.3 | 2.78 ± 0.3 | 2.80 ± 0.3 | 2.73 ± 0.3 |
| control | 2.78 ± 0.3 | 2.72 ± 0.4 | 2.78 ± 0.3 | 2.78 ± 0.3 | 2.72 ± 0.4 | 2.71 ± 0.3 | 2.80 ± 0.3 | 2.78 ± 0.2 |

*P < 0.05 vs. the control group,
**P < 0.01 vs. the control group
In summary, administration of LZ-H or LZ-M resulted in a decrease of intra-ocular pressure in normal rabbits, wherein the intra-ocular pressure started to drop since 120 min after administration, reached the lowest level at 180 min (P < 0.05), and maintained till 360 min. LZ-L administration did not affected the intra-ocular pressure evidently.

1. An ophthalmic, otic or nasal pharmaceutical composition, comprising levofloxacin or the pharmaceutical acceptable salts thereof and loteprednol etabonate, wherein the weight ratio of loteprednol etabonate to levofloxacin is 1:0.2-5.

2. The ophthalmic, otic or nasal pharmaceutical composition according to claim 1, wherein the weight ratio of loteprednol etabonate to levofloxacin or the pharmaceutical acceptable salts thereof is 1:0.5-1.75, preferably 1:1.

3. The ophthalmic, otic or nasal pharmaceutical composition according to claim 1, which is in a form of medication selected from a group consisting of eye drops, suspension-type eye drops, eye gel, eye cream, ear drops, and nose drops.

4. The ophthalmic, otic or nasal pharmaceutical composition according to claim 1, further comprising one or more of pharmaceutical excipient selected from a group consisting of an isotonic regulator, a preservative, a pH regulator, a suspending agent, and a moistening agent.

5. The ophthalmic, otic or nasal pharmaceutical composition according to claim 1, which are suspension-type eye drops or ear drops, nose drops, wherein there contains 0.1-1.0 g loteprednol etabonate and 0.1-1.0 g levofloxacin or the pharmaceutical acceptable salts thereof in each 100 ml solution.

6. The ophthalmic, otic or nasal pharmaceutical composition according to claim 1, which are suspension-type eye drops or ear drops, nose drops, wherein there contains 0.5 g loteprednol etabonate and 0.5 g levofloxacin or the pharmaceutical acceptable salts thereof in each 100 ml solution.

7. The ophthalmic, otic or nasal pharmaceutical composition according to claim 1, which is eye gel or eye cream, wherein there contains 0.5 g loteprednol etabonate and 0.5 g levofloxacin or the pharmaceutical acceptable salts thereof in each 100 g or 100 ml preparation.

8. The ophthalmic, otic or nasal pharmaceutical composition according to claim 6, which are suspension-type eye drops or ear drops, nose drops, wherein there contains 0.5 g loteprednol etabonate, 0.5 g levofloxacin, 0.1-1.5 g boric acid, 0.05-1.7 g borax, 0.01-0.9 g sodium chloride, 0.01-0.5 g poloxamer, 0.01-0.6 g trichloro-tertiary butyl alcohol, 0.01-1 g sodium hyaluronate in each 100 ml solution and the balance is water for injection.

9. The ophthalmic, otic or nasal pharmaceutical composition according to claim 6, which are suspension-type eye drops or ear drops, nose drops, wherein there contains 0.5 g loteprednol etabonate, 0.5 g levofloxacin, 1.2 g boric acid, 0.06 g borax, 0.22 g sodium chloride, 0.05 g poloxamer, 0.3 g trichloro-tertiary butyl alcohol, 0.035 g sodium hyaluronate in each 100 ml solution and the balance is water for injection.

10. The use of ophthalmic, otic or nasal pharmaceutical composition according to any one of claim 1 in preparation of the medication for treatment of conjunctivitis, keratitis, blepharitis, dacrycystitis, hordeolum, corneal ulcer and ocular infection accompanied with ophthalmitis and even inflammation of the surrounding tissues, to prevent increase of bacterial infection risks and the tissue inflammation of the infected area after the ophthalmic surgeries or ocular injuries, to treat or alleviate the bacterial infection in combination with the tissue inflammation of the infected area, or to treat tympanitis, otitis externa and infective rhinitis.

11. The use of ophthalmic, otic or nasal pharmaceutical composition according to any one of claim 2 in preparation of the medication for treatment of conjunctivitis, keratitis, blepharitis, dacrycystitis, hordeolum, corneal ulcer and ocular infection accompanied with ophthalmitis and even inflammation of the surrounding tissues, to prevent increase of bacterial infection risks and the tissue inflammation of the infected area after the ophthalmic surgeries or ocular injuries, to treat or alleviate the bacterial infection in combination with the tissue inflammation of the infected area, or to treat tympanitis, otitis externa and infective rhinitis.

12. The use of ophthalmic, otic or nasal pharmaceutical composition according to any one of claim 3 in preparation of the medication for treatment of conjunctivitis, keratitis, blepharitis, dacrycystitis, hordeolum, corneal ulcer and ocular infection accompanied with ophthalmitis and even inflammation of the surrounding tissues, to prevent increase of bacterial infection risks and the tissue inflammation of the infected area after the ophthalmic surgeries or ocular injuries, to treat or alleviate the bacterial infection in combination with the tissue inflammation of the infected area, or to treat tympanitis, otitis externa and infective rhinitis.

13. The use of ophthalmic, otic or nasal pharmaceutical composition according to any one of claim 4 in preparation of the medication for treatment of conjunctivitis, keratitis, blepharitis, dacrycystitis, hordeolum, corneal ulcer and ocular infection accompanied with ophthalmitis and even inflammation of the surrounding tissues, to prevent increase of bacterial infection risks and the tissue inflammation of the infected area after the ophthalmic surgeries or ocular injuries, to treat or alleviate the bacterial infection in combination with the tissue inflammation of the infected area, or to treat tympanitis, otitis externa and infective rhinitis.

14. The use of ophthalmic, otic or nasal pharmaceutical composition according to any one of claim 5 in preparation of the medication for treatment of conjunctivitis, keratitis, blepharitis, dacrycystitis, hordeolum, corneal ulcer and ocular infection accompanied with ophthalmitis and even inflammation of the surrounding tissues, to prevent increase of bacterial infection risks and the tissue inflammation of the infected area after the ophthalmic surgeries or ocular injuries, to treat or alleviate the bacterial infection in combination with the tissue inflammation of the infected area, or to treat tympanitis, otitis externa and infective rhinitis.

15. The use of ophthalmic, otic or nasal pharmaceutical composition according to any one of claim 6 in preparation of the medication for treatment of conjunctivitis, keratitis, blepharitis, dacrycystitis, hordeolum, corneal ulcer and ocular infection accompanied with ophthalmitis and even inflammation of the surrounding tissues, to prevent increase of bacterial infection risks and the tissue inflammation of the infected area after the ophthalmic surgeries or ocular injuries, to treat or alleviate the bacterial infection in combination with the tissue inflammation of the infected area, or to treat tympanitis, otitis externa and infective rhinitis.

16. The use of ophthalmic, otic or nasal pharmaceutical composition according to any one of claim 7 in preparation of the medication for treatment of conjunctivitis, keratitis, blepharitis, dacrycystitis, hordeolum, corneal ulcer and ocular infection accompanied with ophthalmitis and even inflammation of the surrounding tissues, to prevent increase of bacterial infection risks and the tissue inflammation of the infected area after the ophthalmic surgeries or ocular injuries, to treat or alleviate the bacterial infection in combination with the tissue inflammation of the infected area, or to treat tympanitis, otitis externa and infective rhinitis.

17. The use of ophthalmic, otic or nasal pharmaceutical composition according to any one of claim 8 in preparation of the medication for treatment of conjunctivitis, keratitis, blepharitis, dacrycystitis, hordeolum, corneal ulcer and ocular infection accompanied with ophthalmitis and even inflammation of the surrounding tissues, to prevent increase of bacterial infection risks and the tissue inflammation of the infected area after the ophthalmic surgeries or ocular injuries, to treat or alleviate the bacterial infection in combination with the tissue inflammation of the infected area, or to treat tympanitis, otitis externa and infective rhinitis.

18. The use of ophthalmic, otic or nasal pharmaceutical composition according to any one of claim 9 in preparation of the medication for treatment of conjunctivitis, keratitis, blepharitis, dacrycystitis, hordeolum, corneal ulcer and ocular infection accompanied with ophthalmitis and even inflammation of the surrounding tissues, to prevent increase of bacterial infection risks and the tissue inflammation of the infected area after the ophthalmic surgeries or ocular injuries, to treat or alleviate the bacterial infection in combination with the tissue inflammation of the infected area, or to treat tympanitis, otitis externa and infective rhinitis.
